(19) United States
(12) Patent Application Publication
(10) Pub. No.: US 2018/0207227 A1
(43) Pub. Date: Jul. 26, 2018

(54) Therapeutic and Neuroprotective Peptides

(71) Applicant: Allegro Pharmaceuticals, Inc., San Juan Capistrano, CA (US)

(72) Inventors: Hampar L. Karageozian, San Juan Capistrano, CA (US); John Y. Park, Santa Ana, CA (US); Vicken H. Karageozian, San Juan Capistrano, CA (US)

(21) Appl. No.: 15/874,814

(22) Filed: Jan. 18, 2018

Related U.S. Application Data
(60) Provisional application No. 62/448,300, filed on Jan. 19, 2017.
(60) Provisional application No. 62/500,998, filed on May 3, 2017.

(51) Int. Cl. A61K 38/08; A61K 38/12; A61P 27/02; A61P 25/02

(52) U.S. Cl. CPC A61K 38/08; A61P 25/02; A61P 27/02; A61K 38/12

(57) ABSTRACT

Nonnatural peptides and their methods of use in human or non-human animal subject to cause an effect such as: nuroprotection, protecting against or lessening nerve impairment or damage, treating glaucoma, treating age-related macular degeneration or other inherited or acquired retinal degenerations, enhancing retinal tissue repair, enhancing retinal regenerative therapy through activation of innate immune cells or treating inherited or acquired retinal degeneration

### RELATED APPLICATIONS

This application claims priority to U.S. Provisional Patent Applications No. 62/448,300 entitled Neuroprotective Peptides filed Jan. 19, 2017 and 62/500,998 entitled Therapeutic Peptides and their Mechanisms of Action filed May 3, 2017, the entire disclosures of both such applications being expressly incorporated herein by reference.

### FIELD OF THE INVENTION

The present invention relates generally to the fields of biology and medicine and more particularly to neuroprotective peptides useable to treat nerve damage that results from neurodegenerative or neuropathic diseases (e.g., glaucoma, retinitis pigmentosa, inherited or acquired retinal degenerations, peripheral neuropathy, neurodegenerative central nervous system (CNS) or peripheral disorders), hypoxic insults (e.g., cardiac arrest or stroke) or mechanical injuries (e.g., trauma, spinal cord injuries) as well as useful for enhancing retinal and neurologic tissue repair and retinal and neurologic regenerative therapy through improving immune modulatory function.

### BACKGROUND

Pursuant to 37 CFR 1.71(e), this patent document contains material which is subject to copyright protection and the owner of this patent document reserves all copyright rights whatsoever.

In addition to traumatic nerve injuries and hypoxic insults, various diseases are known to cause neurodegenerative or neuropathic effects. For example, glaucoma is an optic neuropathy that causes excavation or “cupping” of the optic disk, degeneration of retinal ganglion cells, and resultant visual field loss. Because elevated intraocular pressure (IOP) is a major risk factor for progression of glaucoma, many treatment strategies have been aimed at lowering intraocular pressure.

Recent research suggests that the nerve degeneration that occurs in glaucoma may result from a process that is similar to that which occurs following traumatic injury to neurons of the central nervous system (CNS). For example, following a CNS injury, levels of certain neurotoxic substances are seen to increase in the extracellular fluid. Those toxic substances are believed to then cause secondary neuronal damage in addition to the mechanical damage that occurred as a result of the primary trauma. Drugs capable of preventing or diminishing the effects of these neurotoxic substances can be candidates for development not only as ocular neuroprotective agents but also as neuroprotective agents useful in reducing neuronal death or impairment following insult or trauma to other neuronal tissues including the brain and spinal cord. See, Yoles, E., et al.; a2-Adrenoreceptor Agonists Are Neuroprotective in a Rat Model of Optic Nerve Degeneration; Investigative Ophthalmology & Visual Science, Vol. 40, No. 1, pp. 65-73 (January 1999) and Neufeld, A. H., et al.; Inhibition of Nitric-Oxide Synthase 2 by Aminoguanidine Provides Neuroprotection of Retinal Ganglion Cells in a Rat Model of Chronic Glaucoma; Proc. Natl. Acad. Sci. USA 96 (1999).

Applicant is presently developing a synthetic oligopeptide (Luminate®, Allegro Ophthalmics, LLC) which inhibits a number of integrins and, when administered to the eye, can cause vitreolysis, posterior vitreo-retinal detachment (PVD) and is useable for treatment of eye disorders such as wet macular degeneration (WMD), diabetic retinopathy (PDR), diabetic macular edema (DME) and vitreomacular traction (VMT). As described herein, Applicant has discovered that this synthetic oligopeptide also demonstrates neuroprotective effects in a rat model of optic nerve degeneration and, as stated above, may also be effective to prevent or restore other types of nerve damage or degeneration, such as secondary neuronal damage associated with traumatic injuries.

### SUMMARY

In accordance with the present invention, there is provided a method for inducing, in a human or non-human animal subject, an effect selected from: neuroprotection, protecting against or lessening nerve impairment or damage, treating glaucoma, treating age-related macular degeneration or other inherited or acquired retinal degenerations, enhancing retinal tissue repair, enhancing retinal regenerative therapy through activation of innate immune cells or treating inherited or acquired retinal degeneration. Such method comprises administering to the subject a non-natural peptide which causes such effect, in an amount that is effective to cause such effect.

In accordance with the invention, the peptide may comprise Glycinyl-Arginyl-Glycinyl-Cysteic-Threonyl-Proline including any fragment, congener, derivative, pharmaceutically acceptable salt, hydrate, isomer, multimer, cyclic form, linear form, conjugate, derivative or other modified form thereof which causes said effect. Other non-natural peptides which are useable in methods of the present invention may include certain ones of the compounds described in copending U.S. Provisional Patent Application No. 62/521,984 filed Jun. 19, 2017, the entire disclosure of which is expressly incorporated herein by reference.

Still further in accordance with the invention, the method may be carried out to protect against damage to, diminish damage to, or restore function after damage to, the optic nerve and/or retina in a subject who suffers from glaucoma, age-related macular degeneration, dry macular degeneration, or other inherited or acquired retinal degenerations like retinitis pigmentosa.

Still further in accordance with the invention, the method may be carried out to treat a subject who has suffered trauma, mechanical injury or insult (e.g., hypoxic or ischemic insult) to the brain, spinal cord, CNS or peripheral nervous system.

Still further in accordance with the invention, the method may be carried out to treat, or restore diminished function of, the brain or other portion of a subject's nervous system following a nerve or brain damaging event such as illness, injury or insult, including but not limited to a cardiac arrest, stroke, hypoxic or ischemic insult, disease, disorder or trauma.

Still further in accordance with the invention, the method may be carried out to protect against or diminish nerve damage due to a neuropathic or neurodegenerative disease or disorder, whether ocular or systemic.

Still further aspects and details of the present invention will be understood upon reading of the detailed description and examples set forth herebelow.

### BRIEF DESCRIPTION OF THE DRAWINGS

The following detailed description and examples are provided for the purpose of non-exhaustively describing some, but not necessarily all, examples or embodiments of the invention, and shall not limit the scope of the invention in any way.

FIG. 1 is a bar graph comparing number of ganglion cells in each field compared to number of fields examined as described in Example 1 below describes Luminate Treatment compared to control

FIG. 2 is a bar graph comparing Total Number of Cells in all the fields compared to the number of total cells in Luminate treatment and control as described in Example 1 below Luminate treatment and control

FIG. 3 is a bar graph comparing retinal pigment epithelium (RPE) cell counts in control and treated plates as described in Example 2 below (Legend: Cont=Control (BSS) Treatment; Lu=Luminate (Only) Treatment; H2O2 100 μM=Peroxide (Only) Treatment; Lu H2O2 100 μM=.Luminate Followed by Peroxide Treatment).

FIG. 4 is a bar graph comparing retinal Muller cell counts in control and treated plates as described in Example 3 below (Legend: Cont=Control (BSS) Treatment; Lu=Luminate (Only) Treatment; KA=Kianic Acid (Only) Treatment and KA-Lu 500 μM=.Luminate Followed by Kianic Acid Treatment).

FIG. 5 shows Histological photomicrographs of retinal tissue taken from rats treated with control or increasing doses of neurotoxic agent Kainic acid as described in Example 4 below.

FIG. 6 is a bar graph comparing retinal neuronal cell counts in control and treated plates as described in Example 5 below (Legend: Cont=Control (BSS) Treatment; Lu=Luminate (Only) Treatment; KA 100 μM=Kianic Acid (Only) Treatment and Lu-KA 100 μM=.Luminate Followed by Kianic Acind Treatment).

### DETAILED DESCRIPTION

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

Applicant has studied the safety and neuroprotective effects of a compound comprising the non-natural peptide Glycinyl-Arginyl-Glycinyl-Cysteic-Threonyl-Proline having the structural formula of Compound 1 below (also referred to as ALG-1001 or Luminate®, Allegro Ophthalmics, LLC):

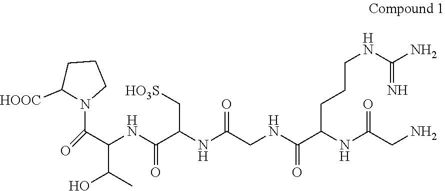 [SMILES: CC(O)C(NC(=O)C(CS(=O)(=O)O)NC(=O)CNC(=O)C(CCCNC(=N)N)NC(=O)CN)C(=O)N1CCCC1C(=O)O]

A cyclic form of non-natural peptide Glycinyl-Arginyl-Glycinyl-Cysteic-Threonyl-Proline is shown below as Compound 2:

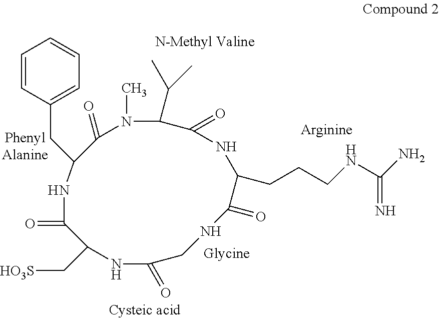 [SMILES: CC(C)C1C(=O)NC(CCCNC(=N)N)C(=O)NCC(=O)NC(CS(=O)(=O)O)C(=O)NC(Cc2ccccc2)C(=O)N1C]

Compounds 1 and 2, as well as other related compounds, are described in copending U.S. patent applications Ser. Nos. 13/1467,995 and 14/696,250, the entire disclosure of each such application being expressly incorporated herein by reference.

### EXAMPLE 1

### Ocular Neuroprotection In Vivo Rat Model of Elevated Intraocular Pressure

Healthy Wister rats (n=8) ten (10) weeks of age were kept in a vivarium maintained at a constant temperature of 26° C., and a constant light-dark cycle (14 hours and 10 hours respectively) with food available ad libitum. The rats were randomly divided into a Luminate treatment group of five (5) animals (Group A) and a Basic Salt Solution (BSS-control) treatment group of three (3) animals (Group B).

The animals of Group A each received a single intravitreal injection of 1.28 mg/20 μL of Luminate in the right eye. The animals of Group B each received a single intravitreal injection of 20 μL of balanced salt solution (BSS). The left eyes of all animals in Groups A and B were not injected and were used as untreated controls. The intravitreal injections were administered 2 mm posterior to the limbus in the supronasal quadrant using a 30-gauge needle attached to a 1.0 cc syringe. Care was taken to avoid damage to the lens or retina.

Twenty-four (24) hours after administration of the injections the rats were anesthetized by intraperitoneal injection of 3.0 mL/kg of a mixture of ketamine Hydrochloride (2.5 mg/mL), diazepam (2.0 mg/mL) and atropine (0.1 mg/mL). The eyes were then subjected to peritoneal conjunctival detachment of the lateral rectus muscle to expose the optic nerve. The optic nerve was then ligated with silk suture for a period of 60 minutes during which the absence of blood flow in the retina of each ligated eye was verified by inspection using a Plano contact lens. The ligatures were removed after 60 minutes, and restoration of blood flow to the retina was verified in each previously-ligated eye using the Plano contact lens.

Following removal of the ligatures and verification that retinal blood flow was restored, the rats were housed alive for 48 hours and then sacrificed by channeling the abdominal aorta and inferior vena cava and perfusion with 200 mL of 10% formaldehyde.

The eyes were then enucleated and fixed for histopathological analysis. Specimens of the retina and optic nerve from each eye were dehydrated and embedded in paraffin. Horizontal sections 4 microns thick were cut and stained with hematoxylin and eosin. Under light microscopy, ganglion cell counts were counted in axial sections of the retina of each eye from ora serrata through the optic nerve. Also, in each section, the number of ganglion cells per millimeter through the total length of the retina was calculated digitally, using a measuring slide calibrated for this purpose.

Measurement of the inner plexiform layer was performed by observing the slides at a magnification of 40× no more than 1 mm from the optic nerve.

The results were subjected to statistical analysis. The results for each group were expressed as mean±standard deviation and the statistical significance between the results of the groups was evaluated by 2 way ANOVA as well as the Mann-Whitney U test. Probabilities of <0.05 were deemed to be significant.

Table 1 below shows the number of ganglion cells per field for five (5) LUMINATE® treated eyes and three BSS-treated (control) eyes:

TABLE 1

| | CASE 1 | CASE 2 | CASE 3 | CASE 4 | CASE 5 |
|---|---|---|---|---|---|
| Field 1 | 20 | 5 | 8 | 16 | 14 |
| Field 2 | 17 | 14 | 10 | 16 | 24 |
| Field 3 | 19 | 25 | 21 | 13 | 20 |
| Field 4 | 19 | 16 | 9 | 28 | 16 |
| Field 5 | 16 | 5 | 23 | 24 | 12 |
| Field 6 | 21 | 25 | 29 | 16 | 15 |
| Field 7 | 22 | 37 | 29 | 15 | 16 |
| Field 8 | 19 | 15 | 31 | 25 | 10 |
| Field 9 | 40 | 16 | 41 | 33 | 12 |
| Field 10 | 28 | 7 | 29 | 8 | 19 |
| Field 11 | 13 | | 29 | 24 | 17 |
| SUM | 234 | 165 | 259 | 218 | 175 |
| SD | 7.268 | 10.157 | 10.596 | 7.454 | 4.036 |

| | Control 1 | Control 2 | Control 3 |
|---|---|---|---|
| Field 1 | 3 | 5 | 4 |
| Field 2 | 6 | 14 | 2 |
| Field 3 | 3 | 25 | 11 |
| Field 4 | 13 | 16 | 15 |
| Field 5 | 3 | 5 | 10 |
| Field 6 | 0 | 25 | 14 |
| Field 7 | 0 | 37 | 15 |
| Field 8 | 1 | 15 | 8 |
| Field 9 | 3 | 16 | 10 |
| Field 10 | 0 | 7 | 19 |
| Field 11 | 2 | | 16 |
| SUM | 34 | 165 | 124 |
| SD | 3.754 | 10.157 | 5.198 |

Table 2, below, displays a two way ANOVA analysis of the data set forth in Table 1. Group A consists of ALG1001-reated eyes and Group B consists of BSS-treated (control) eyes:

TABLE 2

| | Group A | | | | | Group B | | | | |
|---|---|---|---|---|---|---|---|---|---|---|
| Table | CASES | | | | | CONTROLS | | | | |
| Format | A: | A: | A: | A: | A: | B: | B: | B: | B: | B: |
| Grouped | Y1 | Y2 | Y3 | Y4 | Y5 | Y1 | Y2 | Y3 | Y4 | Y5 |
| Field 1 | 20 | 5 | 8 | 16 | 14 | 3 | 5 | 4 | | |
| Field 2 | 17 | 14 | 10 | 16 | 24 | 6 | 14 | 2 | | |
| Field 3 | 19 | 25 | 21 | 13 | 20 | 3 | 25 | 11 | | |
| Field 4 | 19 | 16 | 9 | 28 | 16 | 13 | 16 | 15 | | |
| Field 5 | 16 | 5 | 23 | 24 | 12 | 3 | 5 | 10 | | |
| Field 6 | 21 | 25 | 29 | 16 | 15 | 0 | 25 | 14 | | |
| Field 7 | 22 | 37 | 29 | 15 | 16 | 0 | 37 | 15 | | |
| Field 8 | 19 | 15 | 31 | 25 | 10 | 1 | 15 | 8 | | |
| Field 9 | 40 | 16 | 41 | 33 | 12 | 3 | 16 | 10 | | |
| Field 10 | 28 | 7 | 29 | 8 | 19 | 0 | 7 | 19 | | |
| Field 11 | 13 | | 29 | 24 | 17 | 2 | | 16 | | |

Table 3 shows tabular results of the ANOVA analysis displayed in table 2, indicating a statistically significant difference (p<0.0001) between the Luminate treated eyes (Group A) and the BSS-treated (control) eyes (Group B).

TABLE 3
2way ANOVA Tabular results

| Table Analyzed | | Two-way ANOVA, not RM | | |
|---|---|---|---|---|
| Two-way ANOVA | | Ordinary | | |
| Alpha | | 0.05 | | |
| Source of | % of total | | P value | |
| Variation | variation | P value | summary | Significant? |
| Interaction | 3.810 | 0.9385 | ns | No |
| Row factor | 12.37 | 0.2386 | ns | No |
| Column factor | 22.62 | <0.0001 | **** | Yes |

| ANOVA table | SS | DF | MS | F (DFn, DFd) | P Value |
|---|---|---|---|---|---|
| interaction | 297.7 | 10 | 29.77 | F(10, 64) = 0.4070 | P = 0.9385 |
| Row Factor | 966.7 | 10 | 96.67 | F(10, 64) = 1.321 | P = 02386 |
| Column | 1767 | 1 | 1767 | F(10, 64) = 24.16 | P < 0.0001 |
| factor | | | | | |
| Residual | 4682 | 64 | 73.16 | | |

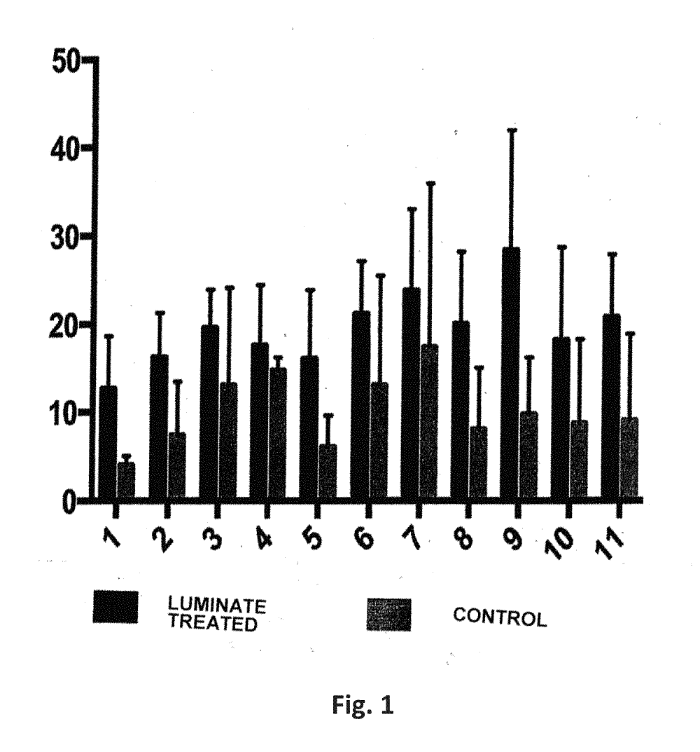

FIG. 1 is a bar graph of number of ganglion cells in each field vs. the number of fields examined for the Luminate treatment and control illustrating the differences between the mean

Table 4 shows tabular results of the Mann-Whitney U Test, which also indicates a statistically significant difference (p<0.0001) between the Luminate-treated eyes (Group A) and the BSS-treated (control) eyes (Group B).

TABLE 4
Mann-Whitney Tabular Results

| | |
|---|---|
| Table analyzed | Data 1 |
| Column B | CONTROLS |
| vs. | vs. |
| Column A | CASES |
| Mann Whitney | |
| test | |
| P value | <0.0001 |
| Exact or | Exact |
| approximate | |
| value? | |
| P value summary | **** |
| Significantly | Yes |
| different?(P < 0.05) | |
| One- or two-tailed | Two tailed |
| value? | |
| Sum of ranks in | 2871, 870.5 |
| column A, B | |
| Mann-Whitney U | 342.5 |
| Difference | |
| between medians | |
| Median of column | 18.00, n = 54 |
| A | |
| Median of column | 9.000, n = 32 |
| B | |
| Difference: Actual | −9.000 |
| Difference: | −10.00 |
| Hodges-Lehmann | |
| 95.05% Cl of | −13.00 to −6.000 |
| difference | |
| Exact or | Exact |
| approximate Cl? | |

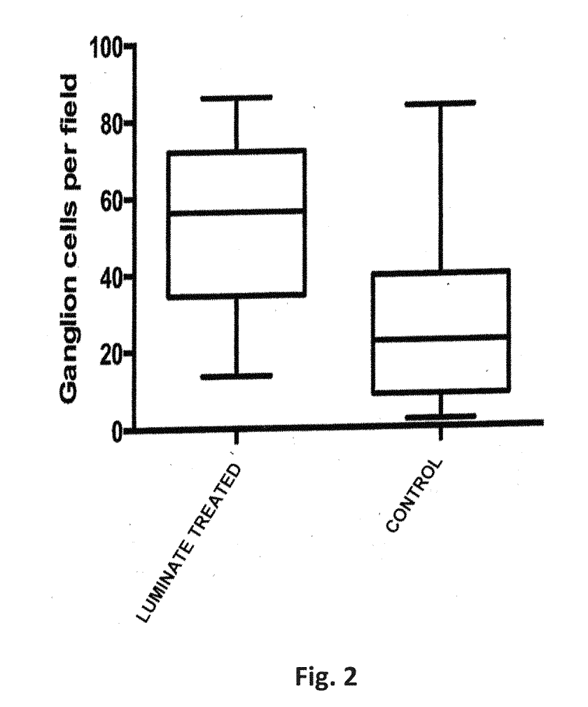

FIG. 2 is a bar graph comparing the mean ganglion cell count per field between the Luminate-treated eyes (Group A) and the BSS-treated (control) eyes (Group B) using a Mann-Whitney U test.

It is concluded from these data of Example 1 that intravitreal administration of a preparation comprising an effective amount of the peptide Glycinyl-Arginyl-Glycinyl-Cysteic-Threonyl-Proline (Luminate) had significant neuroprotective effects in this rat model of elevated IOP. As noted above, positive results in this animal model of glaucoma induced neuronal damage in the eye are not only indicative of utility as an ocular neuroprotective agent but also as a neuroprotective agent useful in reducing neuronal death or impairment following insult or trauma to other neuronal tissues including the brain and spinal cord.

### EXAMPLE 2

### In Vitro Neuroprotective Effects of Luminate on Retinal Pigment Epithelium (RPE)

Hydrogen peroxide (H2O2), a physiological mediator of oxidative stress, is known to induce apoptosis in retinal pigment epithelial (RPE) cells.

ARPE-19 cells were incubated in DMEM/F12 medium supplemented with 10% fetal bovine serum (FBS) and 50 μg/ml streptomycin and 50 μg/ml of penicillin at 37° C. in an atmosphere of 5% CO₂. To induce differentiation, ARP-19 cells were cultured in a Laminin coated transwells for 2 weeks in the same medium supplemented with 1% FBS and antibiotics. The RPE cells were then isolated and aliquots of about 150 μl-200 μl of cell suspension were dispensed into the petri dishes containing control medium. The cells were then incubated at 37 degrees C. for 24 hours before use.

Thereafter, the following four (4) separate neuronal cell dishes were prepared as shown below:

A) Control Retinal RPE cells;

B) Retinal RPE cells incubated with 1.0 mg/ml ALG-1001 (Luminate);

C) Retinal RPE cells incubated with 100 μM of Hydrogen Peroxide; and

D) Retinal RPE cells incubated with 1.0 mg/ml ALG-1001 (Luminate) for 24 hours before exposure to 100 μM of Hydrogen Peroxide.

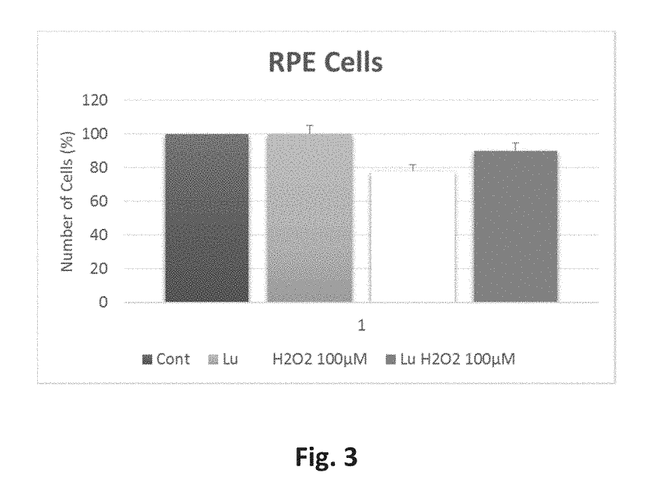

Eight (8) hours post exposure, the cell numbers were measured using Trypan blue exclusion assay in a Neubaur Chamber. FIG. 3 is a bar graph comparing the RPE cell counts in Plates A, B, C and D. These data of Example 2 show that hydrogen peroxide was toxic to the RPE cells, as evidenced by the fact that the RPE cell count in the plate treated with Hydrogen Peroxide alone (Plate C) was only 78% of the control cell count (Plate A). However, the RPE cell count in the plate pretreated with ALG-1001 (Luminate) prior to hydrogen peroxide exposure (Plate D) was 90% of the control cell count (Plate A), thereby indicating that the ALG-1001 (Luminate) pretreatment had a neuroprotective effect in this in vitro model.

### EXAMPLE 3

### In Vitro Neuroprotective Effects of Luminate on Retinal Muller Cells

CD1 mice were euthanized by decapitation, eyes were rapidly enucleated into DMEM complement with antibiotic solution and stored overnight at room temperature. Subsequently the intact globes were incubated in DMEM containing 0.1% trypsin and 70 U/ml collagenase at 37° C. for 60 minutes.

The incubated materials were placed in a petri dish containing DMEM supplemented with 10% fetal bovine serum, and the retinas were removed without the RPE cells into small aggregates and seeded into 35 mm culture dishes. Medium was unchanged for 6 days and then replenished every 3-4 days. The cultures were maintained at 37° C. in a 55% CO₂/95% O₂ in a humidified incubator.

When the cell outgrowth had attained semiconfluency, (80%) retinal aggregates were removed by pipetting the medium into the dish. This operation was repeated three (3) times until a purified flat cell population was obtained. After 24 hours, the cells were exposed to the experimental conditions.

Four (4) separate Muller cell dishes were prepared, as follows: A) Control Muller cells; B) Muller cells incubated with ALG-1001 (Luminate) 1.0 mg/ml; C) Muller cells incubated with 500 μM of Kainic acid and D) Muller cells incubated with Luminate 1.0 mg/ml for 24 hours before exposure to 500 μM of Kainic acid. Forty Eight (48) hours post exposure, the cell numbers were measured using Trypan blue exclusion assay in a Neubaur Chamber.

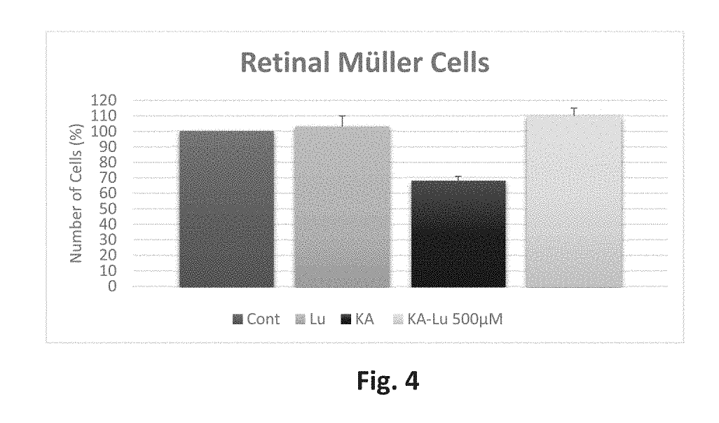

FIG. 4 is a bar graph comparing the Muller cell counts in Plates A, B, C and D. These data indicate that the plate incubated with Luminate 1.0 mg/ml for 24 hours before exposure to 500 μM of Kainic acid (Plate D) had a higher Muller cell count than any of the other plates (A, B or C) and substantially more Muller cells than the plate (Plate C) which received the Kainic acid challenge without Luminate pretreatment.

Forty Eight (48) hours post exposure, the cell numbers were measured using Trypan blue exclusion assay in a Neubaur Chamber. FIG. 4 is a bar graph comparing the Muller cell counts in Plates A, B, C and D. These data indicate that the plate incubated with Luminate 1.0 mg/ml for 24 hours before exposure to 500 μM of Kainic acid (Plate D) had a higher Muller cell count than any of the other plates (A, B or C) and substantially more Muller cells than the plate (Plate C) which received the Kainic acid challenge without Luminate pretreatment.

### EXAMPLE 4

### In Vivo Dose-Related Neurotoxic Effects of Kainic Acid

The right eyes of 4 Wister rats were injected intravitreally with 20 μl of the four different solutions, as follows: A) BSS solution as control, B) 0.5 mM of Kainic acid, C) 5.0 mM of Kainic acid and D) 50.0 mM of Kainic acid.

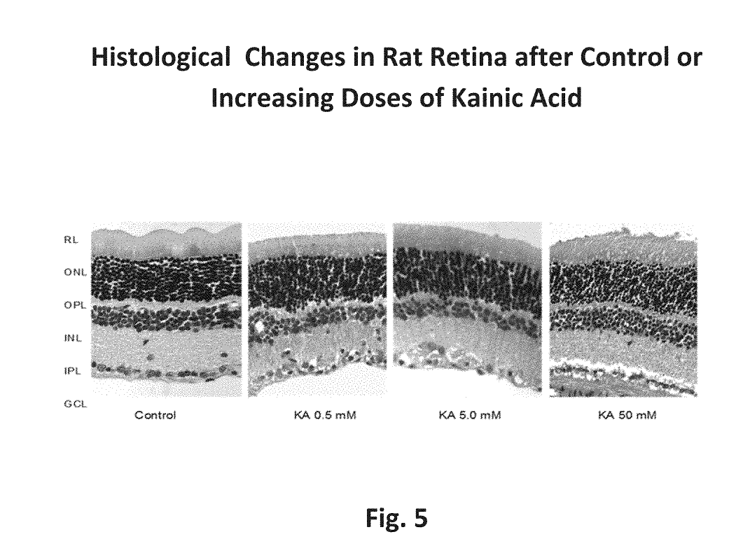

The rats were sacrificed 24 hours post treatment and the eyes were prepared for Histopathology examination. FIG. 5 shows representative histological sections for each of the four (4) treated eyes.

The results clearly demonstrate the degeneration of the Wister rat retina as the concentration of Kainic acid is increased from 0.5 mM to 5.0 mM to 50.0 mM. This confirms that Kainic acid does causes dose-related retinal neurotoxisity in vitro and confirms the relevance of studies using Kainic acid treated retinal cells in In-vitro, such as Examples 2, 3 and 5 of this patent application.

### EXAMPLE 5

### In Vitro Neuroprotective Effects of Luminate on Retinal Neuronal Cells

CD1 mice were euthanized by decapitation, eyes were rapidly enucleated into DMEM complement with antibiotic solution and the retina were isolated from the pigment epithelium.

The isolated retina was incubated in Hank's medium containing 2.5 mg/ml papain and 0.1 mg/ml of cysteine for 15 minutes at 30° C. After rinsing the Hank's medium, supplemented with 1.9 mM of CaCl₂, 0.6 mM of MgCl₂ and 0.1 mg/ml of bovine serum albumin.

The retina was mechanically dissociated, about 150 μl-200 μl of cell suspension was dispensed into the petri dish containing control medium. Bipolar cells were identified under a microscope by the cells morphology. The cells were incubated for 6 hours before use.

Four (4) separate Neuronal cell dishes were prepared, as follows: A) Control Retinal Neuronal cells, B) Retinal Neuronal cells incubated with ALG-1001 (Luminate) 1.0 mg/ml, C) Retinal Neuronal cells incubated with 100 μM of Kainic acid, D) Retinal Neuronal cells incubated with Luminate 1.0 mg/ml for 24 hours before exposure to 500 μM of Kainic acid.

Eight (8) hours post exposure, the cell numbers were measured using Trypan blue exclusion assay in a Neubaur Chamber.

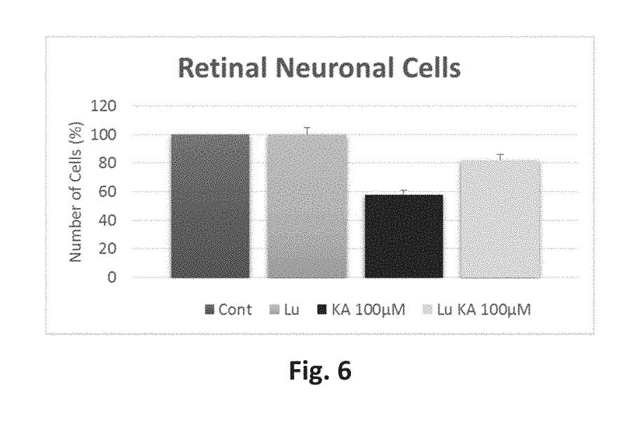

FIG. 6 is a bar graph comparing the retinal neuronal cell counts in Plates A, B, C and D. These data indicate that Kainic acid was toxic to the retinal neuronal cells and Luminate pretreatment substantially lessened that toxicity. Specifically, the neuronal cell count in the plate treated only with 100 μM of Kainic acid)Plate C) was 58% compared to Control while the neuronal cell count in the plate incubated with Luminate 1.0 mg/ml for 24 hours before exposure to 500 μM of Kainic acid (Plate D) was 80% of Control. These results are particularly noteworthy given that Plate D received five (5) times as much Kainic acid as Plate C.

### EXAMPLE 6

### Human, Prospective, Open Label Study for Luminate in the Treatment of Dry AMD

To determine if a single Luminate intravitreal injection of 1.0 mg/50 μL has any effect on improving Best Corrected Visual Acuity (BCVA) of Dry AMD subjects with moderate to moderately sever Dry AMD.

This is a prospective interventional, open label, IRB approved human clinical proof of concept study conducted in 7 human subjects with moderate to moderately severe Dry AMD.

Main inclusion criteria involved patients with Dry Macular Degeneration eyes with relatively intact photoreceptor and RPE layers in the central 1 mm of the macula by OCT.

The baseline BCVA of the subjects was between 20/30 and 20/400 with no evidence of sub-retinal fluid or CNV and no history of Anti-VEGF treatment.

All recruited patients underwent a baseline single intravitreal injection of Luminate 1.0 mg/50μL and were monitored monthly, in addition central macular thickness, OCT, digital color photographs and pre and post treatment BCVA were obtained.

The results of this open label proof of concept study for Luminate in the treatment of Dry AMD in human patients are summarized in Table 5 below:

TABLE 5

| Patient | Baseline BCVA | BCVA 3 Months Post Treatment |
|---|---|---|
| 1 | 0.5 log Mar (20/63) | 0.3 log Mar (20/40) |
| | | Gained 10 letters |
| 2 | 1.0 log Mar (20/200) | 0.6 log Mar (20/80) |
| | | Gained 20 letters |
| 3 | 1.3 log Mar (20/400) | 1.0 log Mar (20/200) |
| | | Gained 15 letters |
| 4 | 0.2 log Mar (20/32) | 1.0 log Mar (20/200) |
| | | Gained 15 letters |
| 5 | 1.3 log Mar (20/400) | 1.3 log Mar (20/400) |
| | | Gained no letters |
| 6 | 0.40 log Mar (20/50) | 0.20 log Mar (20/32) |
| | | Gained 10 letters |

*Patient 5 had was observed to have the worst baseline foveal anatomic features of the group and did not exhibit a detectable improvement in BCVA.

These results indicate that in this study, the BCVA of subjects treated with Luminate improved by up to 20 letters.

It is to be appreciated that, although the invention has been described hereabove with reference to certain examples or embodiments of the invention, various additions, deletions, alterations and modifications may be made to those described examples and embodiments without departing from the intended spirit and scope of the invention. For example, any elements, steps, members, components, compositions, reactants, parts or portions of one embodiment or example may be incorporated into or used with another embodiment or example, unless otherwise specified or unless doing so would render that embodiment or example unsuitable for its intended use. Also, where the steps of a method or process have been described or listed in a particular order, the order of such steps may be changed unless otherwise specified or unless doing so would render the method or process unsuitable for its intended purpose. Additionally, the elements, steps, members, components, compositions, reactants, parts or portions of any invention or example described herein may optionally exist or be utilized in the absence or substantial absence of any other element, step, member, component, composition, reactant, part or portion unless otherwise noted. All reasonable additions, deletions, modifications and alterations are to be considered equivalents of the described examples and embodiments and are to be included within the scope of the following claims.

What is claimed is:

1. A method for inducing in a human or non-human animal subject, an effect selected from: neuroprotection, protecting against or lessening nerve impairment or damage, treating glaucoma, treating age-related macular degeneration or other inherited or acquired retinal degenerations, enhancing retinal tissue repair, enhancing retinal regenerative therapy through activation of innate immune cells or treating inherited or acquired retinal degeneration, said method comprising the step of:
administering to the subject a non-natural peptide which causes said effect, in an amount that is effective to cause said effect.

2. A method according to claim 1 wherein the peptide comprises Glycinyl-Arginyl-Glycinyl-Cysteic-Threonyl-Proline or a fragment, congener, derivative, pharmaceutically acceptable salt, hydrate, isomer, multimer, cyclic form, linear form, conjugate, derivative or other modified form thereof.

3. A method according to claim 2 wherein the composition comprises an effective amount of Compound 1.

4. A method according to claim 2 wherein the composition comprises an effective amount of Compound 2.

5. A method according to claim 1 wherein the method is carried out to protect against or diminish damage to optic nerve and/or retina due to above normal intraocular pressure.

6. A method according to claim 1 wherein the method is carried out to protect against damage to, diminish damage to or restore function after damage to, the optic nerve and/or retina in a subject who suffers from glaucoma, age-related macular degeneration or other inherited or acquired retinal degenerations like retinitis pigmentosa.

7. A method according to claim 1 wherein the method is carried out to treat a subject who has suffered a traumatic or mechanical injury to a nerve.

8. A method according to claim 7 wherein the method is carried out to treat a subject who has suffered a traumatic or mechanical injury to the brain, spinal cord, or peripheral nervous system.

9. A method according to claim 1 wherein the method is carried out in a subject who has suffered trauma or hypoxic insult to the brain.

10. A method according to claim 1 wherein the method is carried out in a subject who has suffered trauma or hypoxic insult to the spinal cord.

11. A method according to claim 1 wherein the method is carried out to protect against or diminish nerve damage due to a neuropathic or neurodegenerative disease or disorder, whether ocular or systemic.

12. A method according to claim 1 wherein the method is carried out to protect against or diminish brain or other nervous system damage following a cardiac arrest, stroke, or hypoxic insult.

13. A method according to claim 1 wherein the method is carried out to restore function after diminished brain or other nervous system damage following a cardiac arrest, stroke, hypoxic insult, or trauma.
